(19) United States
(12) Patent Application Publication
(10) Pub. No.: US 2004/0267347 A1
(43) Pub. Date: Dec. 30, 2004

(54) Protective elongated sleeve for stent systems

(76) Inventor: Marvin John Cervantes, Santa Rosa, CA (US)

Correspondence Address: FRANK C. NICHOLAS, CARDINAL LAW GROUP, Suite 2000, 1603 Orrington Avenue, Evanston, IL, 60201, US

(21) Appl. No.: 10/829,506

(22) Filed: Apr. 22, 2004

Related U.S. Application Data
(60) Provisional application No. 60/466,935, filed on May 1, 2003.

(51) Int. Cl. A61F 2/06

(52) U.S. Cl. 623/1.11; 623/1.1

(57) ABSTRACT

The present invention provides a protective sleeve for a stent assembly, including a catheter and a stent deployment assembly coupled to the catheter. The protective sleeve may either protect the stent assembly from damage from the o-ring of a toughy lock, or may protect the stent assembly until delivered to the target site. Several configurations of the sleeve are disclosed.

### RELATED APPLICATION

This application claims priority to U.S. Provisional Application No. 60/466,935, “Protective Elongated Sleeve for Stent Systems” to Marvin Cervantes, filed May 1, 2003, the entirety of which is incorporated by reference.

### FIELD OF THE INVENTION

This invention relates generally to catheter deployment of drug-coated stents. More specifically, the invention relates to a protective elongated sleeve for stent systems.

### BACKGROUND OF THE INVENTION

An increasing number of stents for treating vascular conditions are being coated with pharmaceutical drugs and protective materials for controlled time-delivery of the therapeutic agents. Medical research indicates a greater effectiveness of vascular stents when the stents are coated with pharmaceutical drugs that help prevent or treat medical conditions. These drugs may be released from a coating while in the body, delivering their patent effects at the site where they are most needed. The drugs may be mixed, for example, within drug-polymers or encased by polymeric coatings on the stents. Stent coatings with various families of drug polymer chemistries have been used to increase the effectiveness of stenting procedures and to control drug-elution properties.

Unfortunately, drug-coated stents, and all other stents, are somewhat fragile, and deployment may lead to several undesirable situations. While inserting the stent through the Y-arm, the stent may contact the inner surfaces of the Y-arm, and may even rub against the o-ring in the toughy. In the case of a drug-eluting stent, such contact may disturb the polymeric surface, and reduce the effectiveness of the covering. While passing through the vasculature, the surface of the stent may also encounter resistance and contact the sidesSuch contact may result in the loss of desired effects at the target site, as the drug elutes through the impacted surface.

It is desirable, therefore, to provide a device that overcomes these and other disadvantages.

### SUMMARY OF THE INVENTION

One aspect of the invention provides a system for treating a vascular condition comprising a catheter, a stent deployment assembly coupled to the catheter and a protective sleeve. The protective sleeve removably covers the stent deployment assembly and at least a portion of the catheter. The protective sleeve comprises a hollow tube and has a proximal outer diameter, a medial inner diameter, and a distal inner diameter. The distal inner diameter is sufficient to encircle an outer diameter of the stent deployment assembly, and the medial inner diameter is sufficient to encircle an outer diameter of the catheter, and wherein the distal inner diameter is open, wherein the protective sleeve is removed from covering the stent framework prior to deploying the stent.

Another aspect of the invention provides a protective sleeve for a drug-eluting stent as part of a stent deployment assembly. The protective sleeve removably covers the stent deployment assembly and at least a portion of the catheter. The protective sleeve comprises a hollow tube and has a proximal outer diameter, a medial inner diameter, and a distal inner diameter. The distal inner diameter is sufficient to encircle an outer diameter of the stent deployment assembly, and wherein the medial inner diameter is sufficient to encircle an outer diameter of the catheter, and wherein the distal inner diameter is open, and the protective sleeve is retracted from covering the stent framework prior to deploying the stent.

In yet another aspect of the invention, a system for treating a vascular condition is provided comprising a catheter and a stent deployment assembly coupled to the catheter; the stent deployment assembly comprising a stent including a stent framework and a drug-polymer coating on at least a portion of the stent framework. The system further comprises means for protecting a surface of the stent framework.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof. The foregoing aspects and other attendant advantages of the present invention will become more readily appreciated by the detailed description taken in conjunction with the accompanying drawings.

### BRIEF DESCRIPTION OF THE DRAWINGS

Various embodiment of the present invention are illustrated by the accompanying figures, wherein:

FIG. 1 is an illustration of a system for treating a vascular condition, in accordance with one embodiment of the current invention;

FIG. 2 is a cross-sectional view of a stent deployment assembly, in accordance with one embodiment of the current invention;

FIG. 3 is a side view of a protective sleeve, in accordance with one embodiment of the current invention;

FIG. 4 is an illustration of a system for treating a vascular condition, in accordance with one embodiment of the current invention;

FIG. 5 is an illustration of a system for treating a vascular condition, in accordance with one embodiment of the current invention;

FIG. 6 is an illustration of a system for treating a vascular condition, in accordance with one embodiment of the current invention;

FIG. 6A is close up view of the distal end of the system illustrated in FIG. 6;

FIG. 7 is an illustration of a system for treating a vascular condition, in accordance with one embodiment of the current invention;

FIG. 8A is an illustration of a system for treating a vascular condition, in accordance with one embodiment of the current invention;

FIG. 8B is a close up view of the distal end of the system illustrated in FIG. 8A;

FIG. 9 is an illustration of a protective sleeve in accordance with one aspect of the invention; and

FIG. 10 is a cross-section of a protective sleeve in accordance with one aspect of the invention.

### DETAILED DESCRIPTION OF THE PRESENTLY PREFERRED EMBODIMENTS

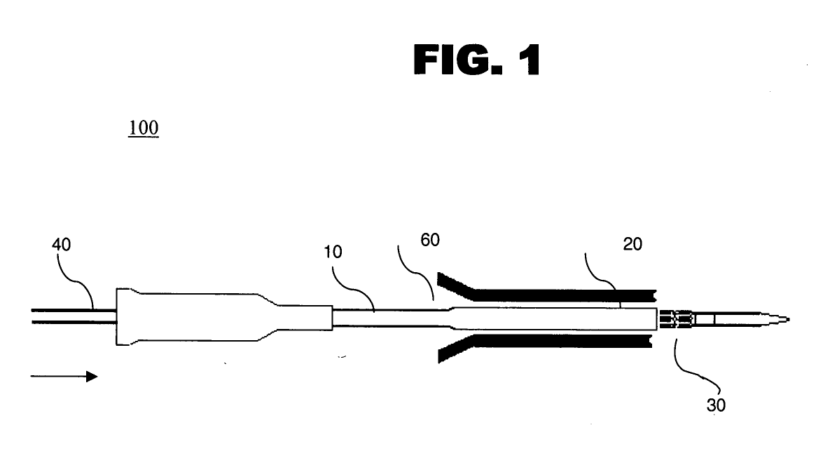

FIG. 1 shows one embodiment of the stent assembly 100. Stent assembly 100 comprises a catheter 40, a protective sleeve 10, a y-arm port 60, and a stent 20. FIG. 1 shows the sleeve 10 partially disposed within a leg of a Y-arm port 60. Y-arm ports are well known to those of ordinary skill in the art. In a currently preferred embodiment, the sleeve 10 is longer than the length of the Y-arm.

In each of the embodiments disclosed herein, the stent includes a stent framework and may comprise a drug coating on at least a portion of the stent framework. The stent framework may comprise a polymeric base or a metallic base such as stainless steel, nitinol, tantalum, MP35N alloy, platinum, titanium, a suitable biocompatible alloy, a suitable biocompatible material, a suitable polymeric material, or a combination thereof. The polymeric base material may comprise any suitable polymer for biomedical stent applications, as is known in the art. In other embodiments, the stent framework may comprise a dissolvable material, such that the stent framework dissolves while implanted in a vessel of a body.

The drug coating may include or encapsulate one or more therapeutic agents. The drug coating may comprise one or more therapeutic agents dispersed within or encased by a polymeric coating, which are eluted from the coated stent with controlled time delivery after deployment of coated stent within a body. A therapeutic agent is capable of producing a beneficial effect against one or more conditions including coronary restenosis, cardiovascular restenosis, angiographic restenosis, arteriosclerosis, hyperplasia, and other diseases and conditions. For example, the therapeutic agent can be selected to inhibit or prevent vascular restenosis, a condition corresponding to a narrowing or constriction of the diameter of the bodily lumen where the stent is placed. The drug coating may comprise, for example, an antirestenotic drug, an antisense agent, an antineoplastic agent, an antiproliferative agent, an antithrombogenic agent, an anticoagulant, an antiplatelet agent, an antibiotic, an anti-inflammatory agent, a steroid, a gene therapy agent, an organic drug, a pharmaceutical compound, a recombinant DNA product, a recombinant RNA product, a collagen, a collagenic derivative, a protein, a protein analog, a saccharide, a saccharide derivative, a bioactive agent, a pharmaceutical drug, a therapeutic substance, or combinations thereof. The elution rates of the therapeutic agents into the body and the tissue bed surrounding the stent framework are based on the constituency and thickness of drug-polymer coating, the nature and concentration of the therapeutic agents, the thickness and composition of cap coat, and other factors.

Protective sleeve 10 is slidably disposed around catheter 40, and stent 20. In one embodiment of the invention, sleeve 10 is fabricated of clear material to allow for viewing a stent 20 contained within the sleeve 10. Alternatively, the sleeve 10 may be fabricated of opaque material to aid in preventing degradation of the coating 30 from light. Alternatively, sleeve 10 may be fabricated of ultraviolet-filtering material for protection from UV radiation. The sleeve 10 may be made, for example, of Teflon or other suitable materials. In other embodiments, sleeve 10 may be made from nylon, polyurethane, polyethylene terephthalate, polyethylene, polytetrafluoroethylene, expanded polytetrafluoroethylene, an elastane, a thermoplastic elastomer, a woven polymeric fabric, or an expandable polymeric sheet.

Drug coating 30 may include one or more drugs, and may comprise a drug-polymer coating. Each drug may include a bioactive agent. The bioactive agent may be a pharmacologically active drug or bioactive compound. The bioactive agent may be eluted from the drug coating 30 when the stent 20 has been deployed in the body. Elution refers to the transfer of the bioactive agent out from drug coating 30. The elution rate is determined by the rate at which the bioactive agent is excreted from drug-polymer coating 30 into the body. The composition of the drug-polymer coating and the interdispersed drugs may control the elution rate of the bioactive agent. The phenoxy primer coating underlying drug coating 30 would tend not to be eluted, metabolized, or discarded by the body.

The drug coating 30 may be subject to degradation during processing, packaging, sterilization, or storage of a drug-polymer coated stent. During sterilization, for example, oxidation of the drug or polymer may occur, resulting in hydrolytic damage, cleavage of the polymeric bonds, breakdown of the polymer and/or drug, or actual cracking or peeling of the drug-polymer coating. Temperature excursions of the in-process or processed stent 20 may incite delamination of all or a portion of the drug coating 30. For stents comprising a dissolvable material, the risks of degradation are increased because of the lack of a metallic structure. The present invention solves this problem through the use of a protective sleeve to reduce or prevent drug-polymer delamination.

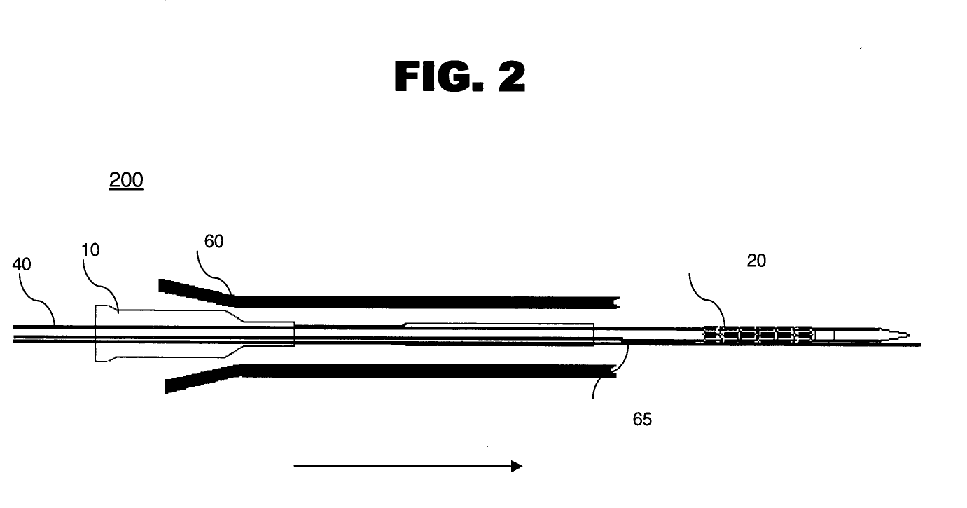

FIG. 2 illustrates stent assembly 200. Stent assembly 200 comprises a catheter 40, a protective sleeve 10, a y-arm port 60, and a stent 20. Stent 20 may comprise a drug coating 30. As seen in FIG. 2, sleeve 10 protects stent 20 from manual handling as stent 20 may be threaded out of sleeve 10 by deploying catheter 40 in the direction of the target site (indicated by arrow). Catheter 40 may protrude distally beyond sleeve 10 thereby providing a means for manipulating stent 20 without handling the stent within sleeve 10. When stent segment 20 is disposed within sleeve 10, sleeve 10 may be handled without handling stent segment 20. In FIG. 1, stent 20 is illustrated partially disposed within the protective sleeve; while in FIG. 2, stent 20 is illustrated not disposed within the protective sleeve 10. Stent segment 20 is therefore, less likely to be disturbed during storage, handling or sleeve placement. Furthermore, sleeve 10 may be handled without disturbing a coating 30 that may be dispersed on stent segment 20. Coating 30 is therefore, less likely to be disturbed during storage, sleeve placement and during lab handling. In embodiments where the stent 20 comprises a dissolvable material, protective sleeve 10 contributes to handling the stent with minimal contact with the actual stent 20.

FIG. 2 shows the sleeve 10 disposed within the Y-arm port 60. In this embodiment, the sleeve 10 will pass through the Y-arm port 60 and will protect the stent 20 until it is removed when the stent 20 is at the desired position. In other embodiments, the outer diameter of sleeve 10 is greater than the inner diameter of an o-ring in the Y-arm, and will therefore not pass through the o-ring. FIG. 1 shows the sleeve 10 prior to entry into a leg of a Y-arm port 60. In a currently preferred embodiment, the sleeve 10 is longer than the length of the Y-arm. FIG. 2 also illustrates a guide wire 65. Guide wires are well known in the art as one method of guiding the stent to its target site.

In both FIG. 1 and FIG. 2, one or more therapeutic agents may be in contact with polymeric coatings on coated stent 20. The system 100 may help treat, for example, heart disease, various cardiovascular ailments, and other vascular conditions by using catheter-deployed endovascular stents that have tailored polymeric coatings for controlling the timed-release properties of interdispersed or encased therapeutic agents. Treatment of vascular conditions may include the prevention or correction of various ailments and deficiencies associated with the cardiovascular system, the cerebrovascular system, urinogenital systems, biliary conduits, abdominal passageways and other biological vessels within the body.

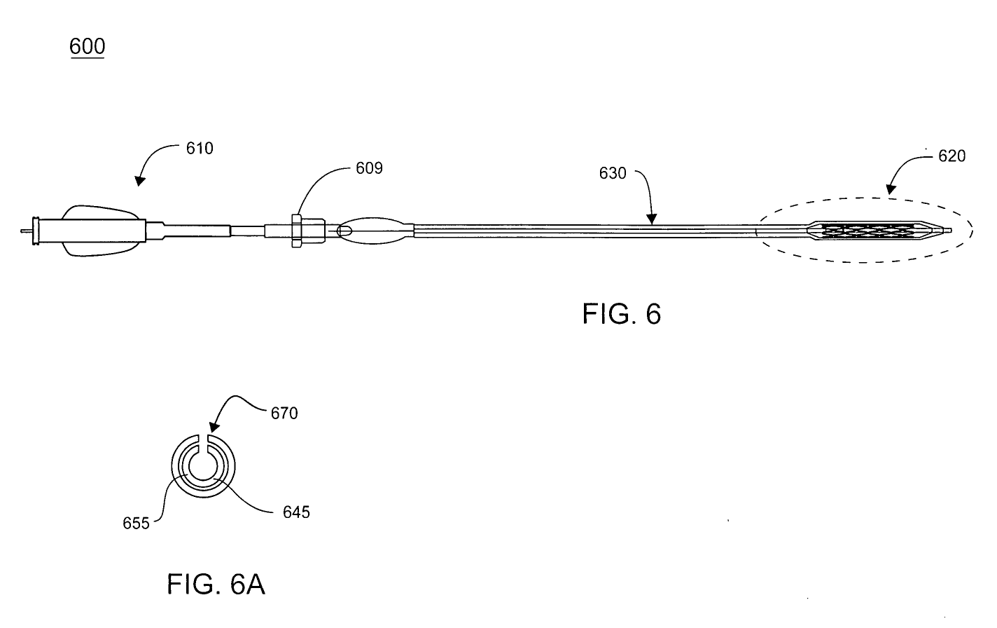

In FIGS. 1 and 2, y-arm 60 generally provides a port to a vessel. Other ports into a vessel are possible, and this invention is adaptable to work with any port into a vessel. Other embodiments, some of which are illustrated in FIGS. 6, 7, and 8, do not use a y-arm port, but use other ports that are generally known in the art.

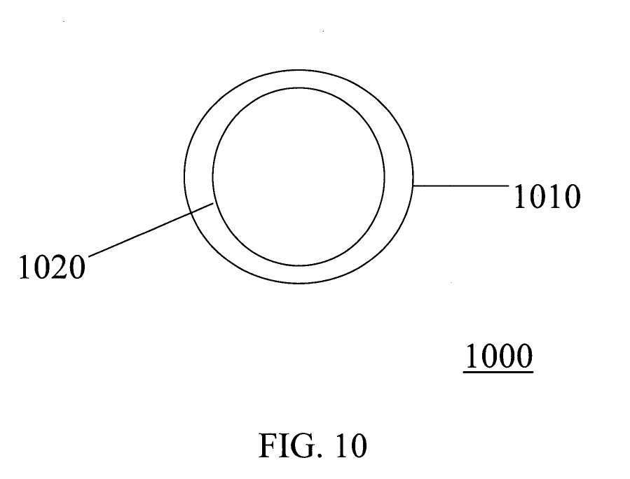

FIG. 10 depicts a cross section of the sleeve at 1000. The outer diameter of the sleeve is depicted as 1010, and the inner diameter of the sleeve is depicted as 1020. In each embodiment of the instant invention the sleeve generally comprises a hollow tube, although the inner and outer diameter may vary along the length of the protective sleeve.

In each of the embodiments disclosed herein, the protective sleeve may comprise a lubricious covering, on the inner surface, the outer surface or both. Thus, referring to FIG. 10, a lubricious covering may cover at least a portion of outer surface 1010, inner surface 1020, or both inner surface 1010 and outer surface 1020. For example, a lubricious coating may be positioned on at least a portion of an outer surface of the protective sleeve. The lubricious coating may comprise a material such as phosphorylcholine, a hydrophilic coating, or a lubricious film. Other portions of the system disclosed herein may also be coated with lubricious coating to minimize the coefficient of friction between the coated surface and structures surrounding the coated surface. For example, a lubricious covering may be applied to the outer surface of the stent and the inner surface of the protective sleeve to ease removal of the protective sleeve at the target location. Patterns, stripes, and fractional coverage of the various surfaces with the lubricious coating may be used to control stent retention during delivery and deployment.

Generally, the outer diameter of the protective sleeve will be determined by determining whether the protective sleeve will be delivered to the vasculature, or whether the protective sleeve should remain in the port to the vessel. In the first embodiment, the sleeve is allowed to pass through the o-ring, and in the second, the sleeve is not allowed to pass through the o-ring. The protective sleeve should have an inner diameter sized to allow free and unrestricted movement of the stent delivery assembly longitudinally, but with limited movement laterally. Optimally, the inner diameter of the sleeve will comprise a lubricious coating to provide for easier removal of the sleeve from the underlying stent delivery assembly. It may also be optimal to construct different sections of the sleeve from different materials. For example, the distal portion of the sleeve may comprise a material that has greater flexibility than the medial or proximal portion of the sleeve.

In embodiments wherein the sleeve has an outer diameter that is greater than the inner diameter of the toughy lock o-ring, the use of the catheter will be generally as used in the prior art. However, where the sleeve is to be removed in the vasculature, the system must also contain means to retract the sheath. Generally these means will be such that the sleeve may be pulled back, with an application of a retracting force at the proximal end of the sleeve. Such means are known in the art, and are in use for self-expanding stents. These means may include the use of wire or rod, or a tubular member. As these means are generally known in the art, the retraction means are not pictured in the figures. This invention is optimally used in conjunction with self-expanding stents, and the means for retracting the sleeve may be used in conjunction with the means for allowing the self-expanding stent to expand.

In the use of a self-expanding stent, it is common that the stent be prevented from expanding at an undesired location with the use of a sheath. Upon reaching the desired deployment site, these self-expanding sheaths are removed. Some embodiments of the protective sleeve disclosed herein may be coupled to a self-expanding stent sheath, although this is not required to practice the invention. Thus, some embodiments may operably connect the retraction means for retaining means of a self-expanding stent with the means to retract the protective sleeve disclosed herein.

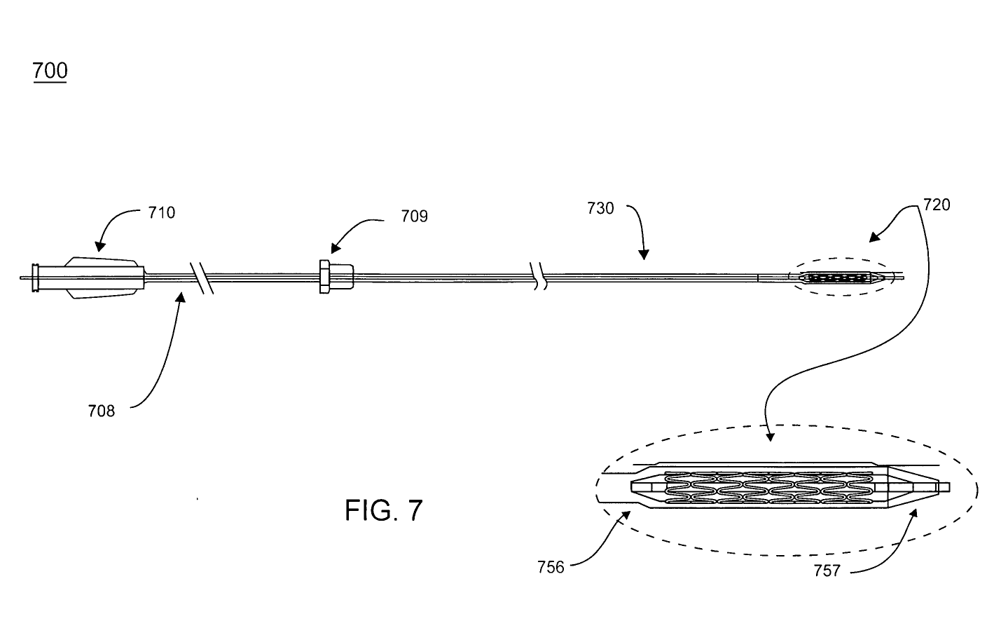

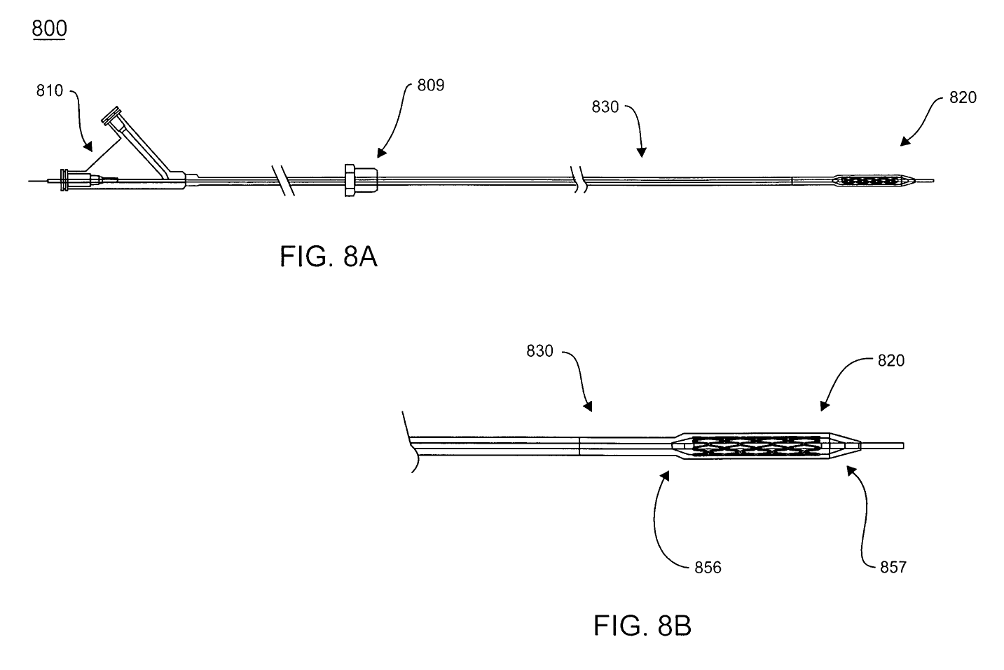

The sleeve is easily adapted to a variety of catheter delivery systems. Example adaptations of the sleeve are depicted in FIG. 6 (Zipper delivery), FIG. 7 (Rapid Exchange delivery), and FIGS. 8A/8B (over the wire). This invention may be used to protect self-expanding stents.

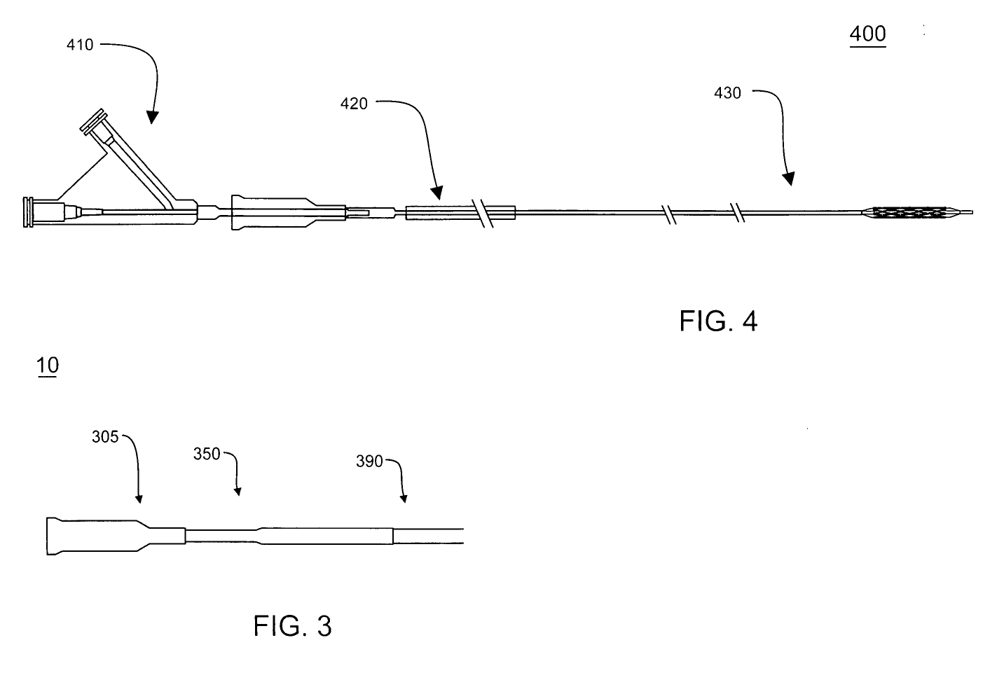

FIG. 3 shows a side view of the protective sleeve 10 in one embodiment of the instant invention. Proximal end 305 tapers to the medial portion 350. Medial portion 350 tapers to the distal end 390. In this embodiment, medial portion 350 has an inner diameter of 0.045 centimeters, and an outer diameter of 0.055 centimeters. The distal end 390 has an inner diameter of 0.071 centimeters, and an outer diameter of 0.0825 centimeters. These measurements are examples only, and any appropriate diameter may be used to practice the invention. In another example, the distal inner diameter will be sufficient to encircle an outer diameter of the catheter, the medial inner diameter will be sufficient to encircle the stent deployment assembly, and the proximal inner diameter will be sufficient to encircle the catheter.

FIG. 4 shows one embodiment of the instant invention. FIG. 4 illustrates stent assembly 400 as shipped. Stent assembly 400 comprises Y-arm 410, protective sleeve 420, and catheter 430. Stent assembly 400 is pictured with the sleeve 420 distal the Y-arm. Catheter 430 is shown surrounded by the sleeve 420.

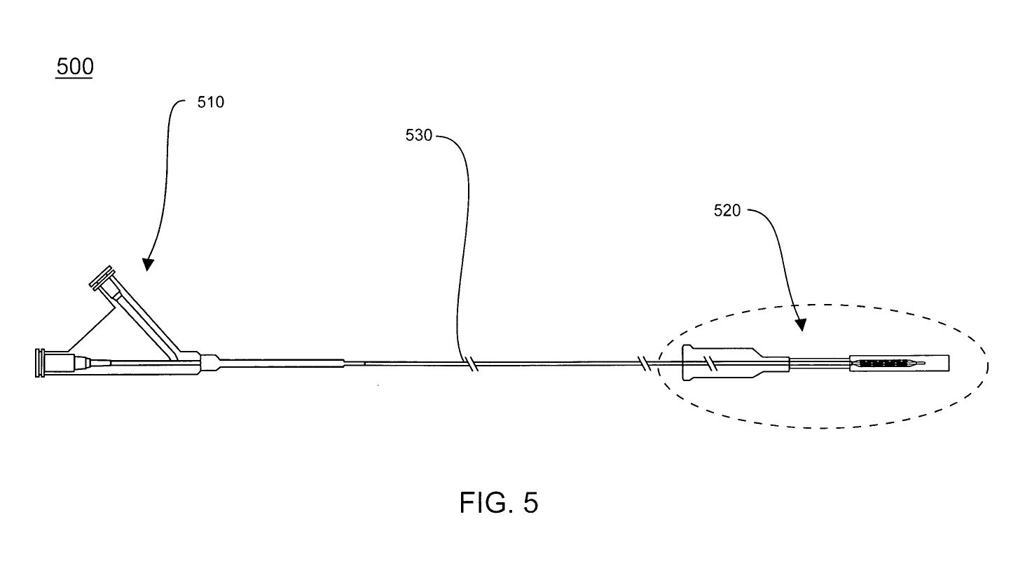

FIG. 5 shows the same embodiment of the instant invention as FIG. 4, but with the sleeve 520 in a deployed position. Stent assembly 500 comprises Y-arm 510, protective sleeve 520, and catheter 530. Sleeve 520 is now distal the position illustrated in FIG. 4, and continues to surround catheter 530. In zipper delivery systems, as illustrated in FIG. 6, the sleeve will comprise a guide wire notch, as is illustrated in greater detail below.

FIG. 6 illustrates an embodiment of the instant invention wherein the sleeve 620 is adapted for use on a zipper delivery system. FIG. 6 shows stent assembly 600, comprising vessel port 610, toughy lock 609, protective sleeve 620, and catheter 630. FIG. 6A is a cross section of the sleeve 620 taken at line B-B. As can be clearly seen in FIG. 6A, when adapted for use on a zipper delivery system, the sleeve 620 comprises an inner shaft 645 and an outer shaft 655. Both the inner shaft 645 and the outer shaft 655 comprise a gap 670 to allow passage of the zipper. The zipper extends longitudinally through gap 670. Referring back to FIG. 6, toughy lock 609 comprises an o-ring (not shown). In embodiments of this invention where the protective sleeve 620 is to remain in the vessel port 610 during deployment, the o-ring will engage the outer diameter of the protective sleeve 620 and prevent the protective sleeve 620 from entering the vasculature. Catheter 630 extends longitudinally from the toughy lock 609, as is known in the art for zipper delivery systems. 610 is a port into a vessel that is not a y-arm.

In another embodiment, the system comprises a guide wire, and the protective sleeve comprises a guide wire notch. The guide wire extends longitudinally through the guide wire notch. The guide wire notch extends at least part of the distance from an outer surface of the protective sleeve through to the inner surface. The protective sleeve slides freely longitudinally along the guide wire riding in the guide wire notch, with substantially no lateral movement.

FIG. 7 illustrates an embodiment of the instant invention for a monorail system. FIG. 7 shows stent assembly 700, comprising vessel port 710, hypotube 708, toughy lock 709, protective sleeve 720, and catheter 730. Catheter 730 extends longitudinally from the hypotube 708, through the toughy lock 709. Protective sleeve 720 is pictured at the distal end of the catheter. The close-up view of the protective sleeve 720 shows the sleeve 720 tapering up at the proximal end 756 of the protective sleeve, and tapering down at the distal end 757 of the protective sleeve. 710 illustrates a port into a vessel that is not a y-arm. FIG. 7 shows an embodiment of the invention wherein the sleeve travels along the catheter to the stent deployment area, and therefore, the protective sleeve 720 is pictured distal the vessel port 710. In a monorail delivery system, the assembly 700 further includes a guidewire (not shown) and the protective sleeve 720 comprises a guidewire notch, wherein the assembly travels longitudinally along the guidewire disposed in the guidewire notch.

FIGS. 8 and 8A illustrate an embodiment of the instant invention utilizing an over-the-wire delivery system (OTW). OTW systems are well known in the art. FIG. 8 shows stent assembly 800, comprising vessel port 810, toughy lock 809, protective sleeve 820, and catheter 830. FIG. 8A shows close-up view of the protective sleeve 820 shows the protective sleeve 820 tapering up at the proximal end 856 of the protective sleeve, and tapering down at the distal end 857 of the protective sleeve. FIG. 8 shows an embodiment of the invention wherein the sleeve travels along the catheter to the stent deployment area, and therefore, the protective sleeve 820 is pictured distal the vessel port 810.

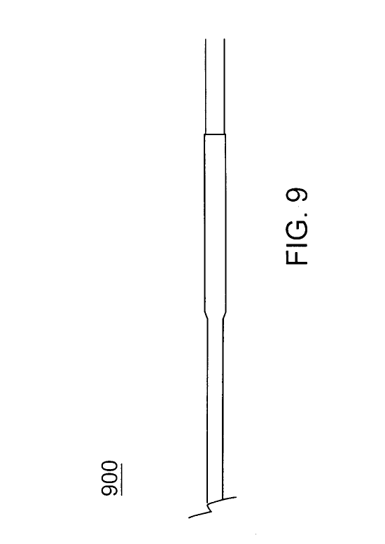

FIG. 9 is a side view of a sleeve 900 in accordance with the instant invention. In this embodiment, the proximal end of the sleeve is smaller than the medial and distal ends of the sleeve. In this example, the proximal end of the sleeve has an inner diameter of 0.045 centimeters, and an outer diameter of 0.055 centimeters. The medial portion and distal portion have an inner diameter of 0.071 centimeters and an outer diameter of 0.0825 centimeters. The medial and distal portions of the sleeve are 7.5 centimeters long as shown, but need only be longer than the Y-arm or other port into a vessel.

While the embodiments of the invention disclosed herein are presently considered to be preferred, various changes and modifications can be made without departing from the spirit and scope of the invention. The scope of the invention is indicated in the appended claims, and all changes that come within the meaning and range of equivalents are intended to be embraced therein.

What is claimed is:

1. A system for treating a vascular condition, comprising:
a catheter;
a stent assembly coupled to the catheter; the stent assembly comprising a coated stent including a stent framework and a drug coating disposed on at least a portion of the stent framework; and
a protective sleeve removably covering the stent deployment assembly and at least a portion of the catheter, wherein said sleeve comprises a hollow tube having a proximal outer diameter, a medial inner diameter, and a distal inner diameter; and wherein the distal inner diameter is sufficient to encircle an outer diameter of the stent deployment assembly, and wherein the medial inner diameter is sufficient to encircle an outer diameter of the catheter, and wherein the distal inner diameter is open, wherein the protective sleeve is removed from covering the stent framework prior to deploying the stent.

2. The system of claim 1 further comprising a port to a vessel, wherein the port comprises a toughy lock, wherein the toughy lock further comprises an o-ring having an o-ring inner diameter, wherein the proximal outer diameter of the sleeve is greater than the o-ring inner diameter.

3. The system of claim 1 further comprising a guide wire, and wherein the sleeve further comprises a guide wire notch, wherein the guide wire extends longitudinally through the guide wire notch.

4. The system of claim 3 wherein the guide wire notch extends at least part of the distance from an outer surface of the sleeve through an inner surface of the sleeve.

5. The system of claim 1 wherein the sleeve comprises a material selected from the group consisting of nylon, polyurethane, polyethylene terephthalate, polyethylene, polytetrafluoroethylene, expanded polytetrafluoroethylene, an elastane, a thermoplastic elastomer, a woven polymeric fabric, or an expandable polymeric sheet.

6. The system of claim 1 wherein the sleeve comprises a material that dissolves while in a vasculature.

7. The system of claim 1 further comprising:
a lubricious coating on at least a portion of a surface of the sleeve.

8. The system of claim 7 wherein the lubricious coating comprises a material selected from the group consisting of phosphorylcholine, a hydrophilic coating, and a lubricious film.

9. The system of claim 1 wherein the sleeve has a distal inner diameter of substantially 0.071 centimeters, a distal outer diameter of substantially 0.0825 centimeters, a medial inner diameter of 0.045 centimeters, and a medial outer diameter of 0.055 centimeters.

10. The system of claim 1 further comprising a port to a vessel, wherein the port comprises a toughy lock, wherein the toughy lock further comprises an o-ring that comprises an o-ring inner diameter, wherein the proximal outer diameter of the sleeve is less than the o-ring inner diameter.

11. A protective sleeve for a stent assembly, comprising:
A hollow tube having a proximal outer diameter, a medial inner diameter, and a distal inner diameter, wherein the distal inner diameter is sufficient to encircle an outer diameter of the stent assembly, and wherein the distal inner diameter is open, and wherein the sleeve retractably covers the stent deployment assembly.

12. The sleeve of claim 11 wherein the sleeve has a distal inner diameter of substantially 0.071 centimeters, a distal outer diameter of substantially 0.0825 centimeters, a medial inner diameter of 0.045 centimeters, and a medial outer diameter of 0.055 centimeters.

13. The sleeve of claim 11 wherein the sleeve has an outer diameter that is greater than the inner diameter of an o-ring of a toughy lock, and wherein the sleeve can not pass the o-ring of the toughy lock during deployment of the stent assembly.

14. The sleeve of claim 11 wherein the sleeve has an outer diameter that is less than the inner diameter of an o-ring of a toughy lock, and wherein the sleeve passes the o-ring of the toughy lock during deployment, and wherein the sleeve is removed from the stent assembly at a site where the stent is to be deployed.

15. The sleeve of claim 11 wherein the sleeve has a distal inner diameter of substantially 0.071 centimeters, a distal outer diameter of substantially 0.0825 centimeters, a medial inner diameter of 0.045 centimeters, and a medial outer diameter of 0.055 centimeters.

16. The sleeve of claim 11 further comprising a lubricious coating on at least a portion of a surface of the sleeve.

17. The sleeve of claim 11 wherein the lubricious coating comprises a material selected from the group consisting of phosphorylcholine, a hydrophilic coating, and a lubricious film.

18. The system of claim 11 wherein the sleeve comprises a material selected from the group consisting of nylon, polyurethane, polyethylene terephthalate, polyethylene, polytetrafluoroethylene, expanded polytetrafluoroethylene, an elastane, a thermoplastic elastomer, a woven polymeric fabric, an expandable polymeric sheet and a material that dissolves while in a vasculature.

19. A system for treating a vascular condition, comprising:
a catheter;
a stent assembly coupled to the catheter; the stent assembly comprising a coated stent including a stent framework and a drug-polymer coating on at least a portion of the stent framework; and
means for protecting a surface of the stent framework.
